(19) United States
(12) Patent Application Publication
(10) Pub. No.: US 2010/0029586 A1
(43) Pub. Date: Feb. 4, 2010

(54) Packaging for a Fabric softening Composition

(76) Inventors: Alexander Schmelz, Milan (IT); Silvana Velasco, Barcelona (ES)

Correspondence Address: UNILEVER PATENT GROUP, 800 SYLVAN AVENUE, AG West S. Wing, ENGLEWOOD CLIFFS, NJ, 07632-3100, US

(21) Appl. No.: 12/226,160

(22) PCT Filed: Mar. 19, 2007
(86) PCT No.: PCT/EP2007/002442

§ 371 Date: Sep. 4, 2009

(30) Foreign Application Priority Data
Apr. 11, 2006 (GB) 0607178.1

(51) Int. Cl. A61K 31/695

(52) U.S. Cl. 514/63

(57) ABSTRACT

A packaged product comprising packaging in combination with a fabric softening composition incorporating a skin benefit agent as an ingredient to enable the fabric softening composition to render textile fabrics treated with the composition capable of delivering the skin benefit agent to the skin with which the fabrics come into contact, characterised in that the packaging incorporates tactile cues (7) to the delivery of skin benefit.

This invention relates to packaging for a fabric softening compositions which incorporates a skin benefit agent such that when fabrics treated with the fabric softening composition subsequently come into contact with the skin the fabrics deliver a benefit to the skin.

Rinse added fabric softener compositions are well known. Typically such compositions contain a water insoluble amine salt and/or a quaternary ammonium fabric softening agent dispersed in water at a level of softening agent up to 7% by weight in which case the compositions are considered dilute, or at levels from 7% to 50% in which case the compositions are considered concentrates. Quaternary ammonium materials with long chain substituents have been used in fabric softening compositions for many years. Often they are used in conjunction with co-actives such as fatty acids or other relatively cheap materials which also provide softening benefits.

Packaging for fabric softeners in the form of bottles are known.

The present invention provides a packaged product comprising packaging in combination with a fabric softening composition incorporating a skin benefit agent as an ingredient to enable the fabric softening composition to render textile fabrics treated with the composition capable of delivering the skin benefit agent to the skin with which the fabrics come into contact, characterised in that the packaging incorporates tactile cues to the delivery of skin benefit.

The present invention also provides a packaged product comprising packaging in combination with a fabric softening composition incorporating a skin benefit agent as an ingredient in to enable the fabric softening composition to render textile fabrics treated with the composition capable of imparting sensory and/or cosmetic benefits to the skin when fabrics treated with the composition come into contact with the skin., characterised in that the packaging incorporates one or more embossment regions to provide tactile cues to the delivery of skin benefit.

The advantageous of such a combination is that the user is encouraged in a wholly intuitive way, to touch the fabrics in order to gain the benefits of the composition.

### The Packaging

The packaging may comprise any suitable container arrangement including a bottle, carton (with or without a liner, or plastic bag inside) or flexible pouch or bag and the like.

The embossment regions may comprise any arrangement of projections and/or indentations on the outer packaging surface. This surface may be the container itself, or it may be labelling materials e.g. adhesive labels, sleeves etc applied to the container.

The projections and/or indentations may be any suitable shape, such as circular, oval etc. The depth of the projections/indentations may be constant or may vary if the embossment region is on a label or thin package, preferably at least 0.05 mm, and maybe between 0.05 and 0.15 mm. However even deeper projections/indentations may be envisaged depending on the suitability (e.g. thickness) of the substrate.

These shapes themselves may contribute to the tactile cue. They may coincide with printed images and/or text and in this way the tactile cue may be enhanced with further visual cues.

The embossed region/s may, either in combination with or alone, present a ‘real’ or lifelike image so as to attract the user to touch the region/s.

The embossed region/s may be provided with other means to attract the user to touch the regions, e.g. bright colours, high reflectivity. These regions may contrast with the background surface. There may be a high gloss finish (e.g. by laquers or the like)applied to the projected areas of a label.

The perforations themselves may bear decorative artwork (shapes, text, images etc.), or may arranged to present this decorative artwork.

There may be solely raised projections from the outer packaging surface. Alternatively there may be solely indentations on this outer surface, each of these arrangements providing a different tactile experience.

Alternatively, there may be combinations of indentations and projections.

The packaging may comprise one or more discrete embossment regions. For example there may be palm-fitting and/or finger-fitting and/or finger-tip areas of the packaging may be provided with embossment regions. The embossment regions may be in a region not normally associated with providing a handle feature.

### Skin Benefit Agents

The skin benefit agents are preferably substantive to fabric. A skin benefit agent is suitably any agent intended to be deposited onto the skin for the purpose of imparting sensory and/or cosmetic benefits thereto. It is preferred if the skin benefit agent changes the sensory perception of the skin. Examples of suitable skin benefit agents include moisturisers, skin softeners, emollients and sunscreens.

The skin benefit agent may be a liquid or solid provided that it is compatible with other ingredients of the fabric softening composition.

Preferred skin benefit agents include:

a) waxes such as carnauba, spermaceti, beeswax, lanolin and derivatives thereof;

b) hydrophobic plant extracts;

c) hydrocarbons such as squalene and squalane;

d) higher fatty acids such as those having at least 12 carbon atoms, for example, lauric, myristic, palmitic, stearic, behenic, oleic, linoleic linolenic, lanolic, isostearic and poly unsaturated fatty acids (PUFA) acids;

e)-higher fatty alcohols such as those having at least 12 carbon atoms, for example, lauryl, cetyl, stearyl, oleyl, behenyl, cholesterol and 2-hexadecanol alcohol;

f) esters such as cetyl octanoate, lauryl lactate, myristyl lactate, cetyl lactate, isopropyl myristate, myristyl myristate, isopropyl palmitate, isopropyl adipate, butyl stearate, decyl oleate, cholesterol isostearate, glycerol monostearate, glyceroldistearate, glycerol tristearate, alkyl lactate, alkyl citrate and alkyl tartrate;

g) essential oils such as fish oils, mentha, jasmine, camphor, white cedar, bitter orange peel, ryu, turpentine, cinnamon, bergamont, citrus unshiu, calamus, pine, lavender, bay, clove, hiba, eucalyptus, lemon, starflower, thyme, peppermint, rose, sage, menthol, cineole, eugenol, citral, citronelle, borneol, linalool, geraniol, evening primrose, camphor, thymol, spirantol, pinene, limonene and terpenoid oils;

h) lipids such as cholesterol, ceramides, sucrose esters and pseudo-ceramides as described in EP-A-556 957;

i) vitamins for sensory and/or cosmetic use such as vitamins A and E, and vitamin alkyl esters, including vitamin C alkyl esters;

j) sunscreens such as octyl methoxyl cinnamate (Parsol (Trade Mark) MCX) and butyl methoxy benzoylmethane (Parsol (Trade Mark) 1789);

k) phospholipids; and

l) derivatives of alpha hydroxy acids such as materials of formula:

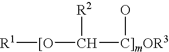

wherein:

R¹ is CₚH_{q}NᵣOₛ, where P is 0-20, q is 1-41, r is 0-3, and s is 0-3;

R² is CₜHᵤ where t is 0-20 and u is 1-41;

R³ is CᵥH_{w}NₓO_{y} where v is 0-20, w is 1-41, x is 0-3 and y is 0-3 or a metallic, ammonium or alkanolammonium anion; and m is 1-10;

m) perfumes;

n) germicides for sensory and/or cosmetic use such as synthetic antimicrobials examples of which include salicylic acid; 1,6 bis (N-p-chlorphenyl biguanido)hexane (Chlorhexidine); chlorhexidine gluconate; 2,4,4-trichloro-2-hydroxy diphenyl ether (Irgosan DP300); imidazolidinyl urea; methyl, propyl, butyl, heptyl and benzyl p hydroxy benzoate; 2-bromo 2-nitropropane-1,3-diol; nonyl phenol ethoxylate iodine complex; 2 phenoxy ethanol; 3-dimethylol-5,5-dimethyl hydantoin; and natural antimicrobials examples of which include willow extract, neem tree extract; bamboo extract; and grapefruit extract;

o) mixtures of any of the foregoing components.

It is preferred if the skin benefit agents are perceived to condition the skin.

A particularly preferred skin benefit agent is a silicone. Examples of preferred silicones are given in GB 1 549 180 (Procter & Gamble).

It is advantageous if the silicone is essentially a linear di(C1-C5)alkylpolysiloxane or (C1-C5) alkylarylpolysiloxane) Examples of such silicones include the polydimethylsiloxanes.

A second class of preferred skin benefit agents comprises are long chain hydrocarbons and esters as disclosed in EP 0 000 406 (Procter & Gamble).

It is preferred if the level of skin benefit agent is from 1 to 25 wt % of the total composition, more preferably from 1 to 10 wt %.

The fabric conditioning composition for use with this invention comprises a fabric softening material. Preferably the fabric conditioning material is a quaternary ammonium softening material. Advantageously the fabric softening composition comprises a water insoluble cationic softening material which is a compound having two C12-28 alkyl or alkenyl groups connected to the N atom via one or more ester links.

A preferred type of ester-linked quaternary ammonium fabric softening material for use in the compositions according to the invention can be represented by the formula:

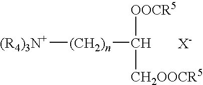

in which each R⁴ group is independently selected from C₁₋₄ alkyl, hydroxyalkyl groups or C₂₋₄ alkenyl groups; and wherein each R⁵ group is independently selected from C₈₋₂₈ alkyl or alkenyl groups; X— is any suitable anion and n is o or an integer from 1 to 5.

Materials of this class and their method of preparation are described in U.S. Pat. No. 4,137,180 (Lever Brothers). Analysis of such materials shows that they also comprise small amounts of the corresponding dimethylamine salt, one such salt being N,N-dimethyl-2,3-bis [hardened tallowoyloxy]-propylamine hydrochloride. Advantageously these materials comprise small amounts of the corresponding monoester as described in U.S. Pat. No. 4,137,180, for example, 1-hardened tallowoyloxy-2-hydroxy-3-trimethylammonium propane chloride.

A further preferred cationic softener is represented by the formula:

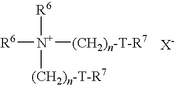

wherein each R⁶ group is independently selected from C₁₋₄ alkyl, hydroxyalkyl or C₂₋₄ alkenyl groups; and wherein each R⁷ group is independently selected from C₈₋₂₈ alkyl or alkenyl groups; T is

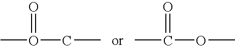

and n is o or an integer from 1 to 5 and X— is any suitable anion.

A further advantage of using ester linked quaternary ammonium materials with the above formula is that included within a composition according to the invention the compositions have excellent viscosities and are stable on storage.

Preferably the level of ester linked quaternary ammonium compounds is at least 1% by weight of the composition, more preferably at least 3% by weight of the composition; especially interesting are concentrated compositions which comprise at least 7% of ester-linked quaternary ammonium compound. The level of ester-linked quaternary ammonium compounds preferably is from 1% to 80% by weight, more preferably from 4% to 32%, most preferably from 6% to 22%.

It is preferable if the ratio of fabric softening compound to skin benefit agent is from 5:1 to 1:5, more preferably from 4:1 to 2:3.

The softening composition may also comprise a nonionic stabilising agent selected from:

i. linear C8 to C22 alcohols alkoxylated with 10 to 20 moles of alkylene oxide; and

ii. a C10 to C20 alcohol or mixtures thereof.

Advantageously the nonionic stabilising agent is a linear C8 to C22 alcohol alkoxylated with 10 to 20 moles of alkylene oxide. Preferably the nonionic stabiliser has an HLB value of from 10 to 20, more preferably from 12 to 20.

Preferably, the level of nonionic stabiliser is within the range from 0 to 10% by weight, more preferably from 0 to 5% by weight, most preferably from 0 to 4% by weight. When nonionic stabilising agent is present the mole ratio of the quaternary ammonium compound to the nonionic stabilising agent is within the range from 40:1 to about 1:1, preferably within the range from 18:1 to about 3:1.

Preferably the compositions of the invention are liquids comprising an aqueous base.

The composition can also contain a co-active, for example, a C8-C24 alkyl or alkenyl monocarboxylic acid or polymer thereof. Preferably saturated fatty acid are used, in particular, hardened tallow (C16-C18) fatty acids. Preferably the fatty acid is non-saponified, for example free oleic acid, lauric acid or tallow fatty acid. Lanolin or other nonionic fabric softening agents may also be used as co-actives.

The level of co-active material is preferably more than 0.1% by weight, more preferably more than 0.2% by weight.

Especially preferred are concentrates comprising from 0.5 to 20% by weight of co-active, more preferably 1% to 10% by weight. The weight ratio of quaternary ammonium/amine material to co-active material is preferably from 10:1 to 1:10.

The composition can also contain one or more optional ingredients, selected from non-aqueous solvents, pH buffering agents, perfumes, perfume carriers, fluorescers, colourants, hydrotropes, antifoaming agents, soil release agents, enzymes, optical brightening agents, opacifiers, anti-shrinking agents, anti-spotting agents, germicides, fungicides, anti-oxidants, anti-corrosion agents, and antistatic agents.

An non-limiting embodiment of the invention will now be described in detail with reference to the accompanying drawings, in which:

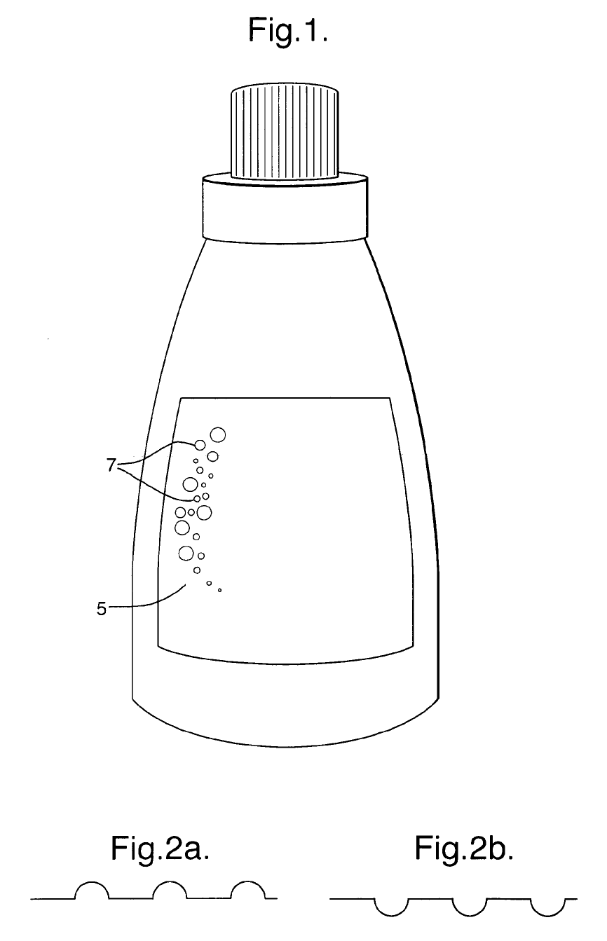

FIG. 1 is a perspective view of a container according to one embodiment of the present invention; and

FIGS. 2a-2b show various embodiments of embossment regions according to the invention including those of the bottle of FIG. 1.

Referring to the FIG. 1 a first embodiment shows a packaged product 1.

The packaged product 1 comprises a labelled bottle 3 in combination with a fabric softening composition (A or 1 or 2 from Table 1 below). The composition incorporates a skin benefit agent as an ingredient to enable the fabric softening composition to render textile fabrics treated with the composition capable of delivering the skin benefit agent to the skin and imparting sensory and/or cosmetic benefits to the skin with which the fabrics come into contact.

The packaging incorporates tactile cues to the delivery of skin benefit.

The bottle is stretch blow-molded. In this case, the body is obtained by blow-moulding of a PET preform or parison. The blowing pressure is comprised between 3 0 and 4 0 bar. The outside of the container presents a single embossment region 5.

The embossment region 5 comprises a plurality of individual projections 7 arranged dynamically on the bottle label. These are circular in plan view, and correspond with graphics (not shown) indicating the skin benefit (e.g. perfume or moisturising or softening). The projections vary and are approximately between 0.05 and 0.15 mm.

The embossment region 5 does not correspond with any handle zone. When the user touches the embossment region, a tactile cue is provided to the skin benefit.

FIG. 2a shows the projections of the embossment region above bottle label, in cross section.

Another embodiment consists of the bottle of FIG. 1 but includes indentations in place of the projections. FIG. 2b shows these indentations in cross section.

A further embodiment consists of the bottle of FIG. 1 but includes as an addition, indentations.

### Composition Examples

In the examples all percentages are expressed by weight.

Rinse conditioner formulations are provided in the Table 1. The were prepared by melting the cationic compound in the silicone fluid with rapid stirring, demineralised water at 70° C. Stirring was continued for 10-15 minutes prior to subjecting the mixture to high shear agitation using a Silverson homogeniser for 10-15 minutes.

TABLE 1

| Example | A | 1 | 2 |
|---|---|---|---|
| HEQ¹ | 5 | 4 | 4 |
| PDMS 5 Pa · S (5000 cSt)² | | 1 | |
| PDMS 30 Pa · S (30,000 cSt)² | | | 1 |

| | |
|---|---|
| Water and minors | To 100 |

¹1,2-bis[hardened tallowoyloxy]-3-trimethylammonium propane chloride/hardened tallow fatty acid - 6:1 quat:fatty acid, ex Hoechst.
²Polydimethylsiloxane silicone fluid, various viscosities, ex Dow Corning.

These formulations deliver a softening benefit, transferring a significant level of silicone onto the skin via the fabrics.

1. A packaged product comprising packaging in combination with a fabric softening composition incorporating a skin benefit agent as an ingredient to enable the fabric softening composition to render textile fabrics treated with the composition capable of delivering the skin benefit agent to the skin with which the fabrics come into contact, characterised in that the packaging incorporates tactile cues to the delivery of skin benefit, and the skin benefit agent comprises a moisturiser.

2. A packaged product comprising packaging in combination with a fabric softening composition incorporating a skin benefit agent as an ingredient to enable the fabric softening composition to render textile fabrics treated with the composition capable of imparting sensory and/or cosmetic benefits to the skin when fabrics treated with the composition come into contact with the skin, characterised in that the packaging incorporates one or more embossment regions to provide tactile cues to the delivery of skin benefit, and the skin benefit agent comprises a moisturiser.

3. A packaged product according to claim 1 wherein the package is a bottle.

4. A packaged product according to claim 1 wherein the one or more embossed regions are provided on a label.
